(19) United States
(12) Patent Application Publication
(10) Pub. No.: US 2021/0401880 A1
(43) Pub. Date: Dec. 30, 2021

(54) Pharmaceutical Composition For Preventing Or Treating Obesity, Containing CYCLO(HIS-PRO) As Active Ingredient

(71) Applicant: Novmetapharma Co., Ltd., Seoul (KR)

(72) Inventors: Heon Jong Lee, Incheon (KR); Kay Olmstead, Escondido, CA (US); Moon Ki Song, Northridge, CA (US); Kyong-Tai Kim, Gyeongsangbuk-do (KR); In-Kyu Lee, Daegu (KR); Hoe-Yune Jung, Gyeongsangbuk-do (KR)

(73) Assignee: Novmetapharma Co., Ltd., Seoul (KR)

(21) Appl. No.: 17/292,238

(22) PCT Filed: Nov. 5, 2019
(86) PCT No.: PCT/KR2019/014932

§ 371 Date: May 7, 2021

(30) Foreign Application Priority Data
Nov. 7, 2018 (KR) 10-2018-0136015

(51) Int. Cl. A61K 33/30; A61K 38/05; A61P 3/10; A61P 3/04

(52) U.S. Cl. CPC A61K 33/30; A61P 3/04; A61P 3/10; A61K 38/05

(57) ABSTRACT

The present invention relates to a pharmaceutical composition for preventing or treating obesity, comprising cyclo-hispro as an active ingredient. Specifically, the present invention has the effect of reducing body weight and body mass, and further improving leptin resistance. In addition, the present invention relates to a pharmaceutical composition for preventing or treating obesity, characterized in that the pharmaceutical composition comprises cyclo-hispro and zinc and is administered once a day.

### TECHNICAL FIELD

The present invention relates to a pharmaceutical composition for preventing or treating obesity, comprising cyclo-hispro as an active ingredient. Specifically, the cyclo-hispro of the present invention has the effect of reducing body weight and body mass, and further improving leptin resistance.

### BACKGROUND ART

Due to the recent improvement in income level and the development of industry, as lifestyles such as dietary life and eating habits are rapidly westernized, patients with chronic diseases and adult diseases are rapidly increasing, and obesity is known to be one of the causes.

Obesity is an abnormality in energy metabolism caused by an imbalance between energy intake and energy consumption, and as a result, it is defined as an excessive accumulation of triglycerides in fat cells.

Obesity is a chronic disease that is a worldwide problem, and there is no effective treatment method, and it is a serious disease that continues to increase. Obesity, unlike other diseases, is characterized by not only an appearance problem, but also being accompanied by associated diseases such as metabolic disease, hypertension, diabetes, hyperlipidemia, arteriosclerosis, ischemic heart disease, fatty liver, and cholelithiasis as well as weight gain.

Obesity is a serious disease in itself, but it also causes cosmetic problems, so many countries around the world have tried to develop various anti-obesity therapeutic agents, and several anti-obesity therapeutic agents have been developed.

Since most anti-obesity therapeutic agents that are currently commercially available have serious side effects, there is a very high social demand for anti-obesity therapeutic agents that can effectively treat obesity without side effects. Accordingly, various studies are being conducted in countries around the world to develop anti-obesity therapeutic agents that can treat obesity without side effects, but such anti-obesity therapeutic agents have not been developed and marketed until now.

Fenfluramine, an anti-obesity therapeutic agent that led the early market, was discontinued for sales in 1997 due to side effects of heart disease and elevated blood pressure. Sibutramine (Meridia® and Reductil® from Abbott), an appetite suppressant, as a representative anti-obesity therapeutic agent was also discontinued for sales in 2009 due to side effects such as increased heart attack.

There is Orlistat (Xenical® and Alli® from Roche) as a drug that has entered the market after clinical trials among anti-obesity therapeutic agents of the lipolysis inhibitor family. Orlistat is an inhibitor of pancreatic lipase, a lipolytic enzyme secreted from the small intestine, and plays a role in inhibiting lipolysis. However, when taking Orlistat, undigested fat moves along the gastrointestinal tract and causes diarrhea and fatty stool and the like, which are not only uncomfortable, but also side effects that make normal social life difficult. In addition, long-term use of Orlistat has been reported to cause serious liver damage in some cases.

In addition, products classified as psychotropic drugs exhibit serious side effects including depression, insomnia, digestive disorders and the like, and long-term use thereof is prohibited.

Liraglutide (Saxenda® from Novonordisk), an anti-obesity therapeutic agent approved as an analog of GLP-1 (Glucagon-Like Peptide 1), was approved as an adjuvant therapy for a low-calorie diet for body weight control and exercise, and is administered by injection once a day. This drug was approved for use in adults having a body mass index (BMI) of 30 or higher (obesity), or adults having a BMI of 27 or higher (overweight) with obesity, hypertension, type 2 diabetes, or hypercholesterolemia (dyslipidemia). However, there are still problems of safety and side effects.

Korean Patent Laid-Open Publication No. 2001-0022786 discloses a composition comprising zinc ion and cyclo-hispro as a composition for treating diabetes mellitus in mammals. However, the above invention is merely to confirm the change in glucose concentration by administering a composition of zinc ion and cyclo-hispro to the animal, and failed to confirm whether it has a pharmacological effect to the extent in which it can be applied to the human body to prevent or treat the disease, specifically whether it has an effect of treating or preventing obesity, and its effective content.

One of the most important tasks in drug administration is to find a dosage and a method of administration that have few side effects and are consistently efficacious. It is very complicated to find the optimal dosage due to the serum half-life, the relationship between dosage and efficacy, especially the correlation with other therapeutic agents for mellitus that are previously prescribed and the like.

Accordingly, the present inventors developed a composition for treating and preventing obesity, comprising cyclo-hispro or a pharmaceutically acceptable salt thereof, which exhibits an excellent anti-obesity effect and has few or less side effects even when taken for a long period of time, thereby completing the present invention.

### DETAILED DESCRIPTION OF THE INVENTION

### Technical Problem

The present invention is to provide a pharmaceutical composition for preventing or treating obesity, comprising cyclo-hispro or a pharmaceutically acceptable salt thereof, which has few or less side effects even when taken for a long period of time. In particular, the present invention is to provide an effective method for preventing and treating obesity by providing a dosage and a method of administration of cyclo-hispro or a pharmaceutically acceptable salt thereof that significantly reduces body weight and improves leptin resistance.

### Solution to Problem

In order to achieve the above object, the present invention relates to a pharmaceutical composition for preventing or treating obesity, comprising cyclo-hispro or a pharmaceutically acceptable salt thereof as an active ingredient.

“Cyclo-hispro (cyclo(his-pro))” is a naturally occurring cyclic dipeptide that has a structure of formula I below and is composed of histidyl and proline.

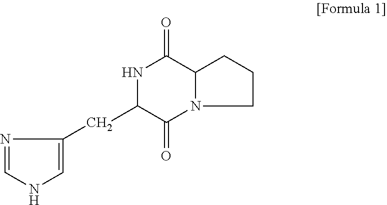 [SMILES: O=C1NC(Cc2c[nH]cn2)C(=O)N2CCCC12]

In addition, the present invention provides a pharmaceutical composition for preventing or treating obesity, characterized in that cyclo-hispro or a pharmaceutically acceptable salt thereof is administered at an amount of 1 mg to 25 mg per day.

In addition, the cyclo-hispro or pharmaceutically acceptable salt thereof may be administered at an amount of 3 mg to 20 mg per day, preferably may be administered at an amount of 6 mg to 15 mg per day, and most preferably may be administered at an amount of 15 mg per day.

The composition may be administered once or several times a day, and preferably may be administered orally once a day.

The present invention provides a pharmaceutical composition for preventing or treating obesity, comprising cyclo-hispro or a pharmaceutically acceptable salt thereof and zinc as an active ingredient.

Specifically, the daily dose of cyclo-hispro or a pharmaceutically acceptable salt thereof may be 1 mg to 25 mg, and the daily dose of zinc may be 15 mg to 30 mg.

### Effect of Invention

The present invention provides a method for preventing or treating obesity comprising cyclo-hispro or a pharmaceutically acceptable salt thereof as an active ingredient, and having few side effects caused by a drug even when taken for a long period of time.

In addition, it can substantially improve and treat obesity by reducing body weight and body mass and reducing plasma leptin level over a long period of time.

In addition, the pharmaceutical composition of the present invention comprises cyclo-hispro or a pharmaceutically acceptable salt thereof, and the present invention provides an effective administration method and dosage for the prevention and treatment of obesity.

### BRIEF DESCRIPTION OF DRAWINGS

FIG. 1 shows the change in concentration (%) of HbA1c over time over 12 weeks for each administration group.

FIG. 2 shows the change in body weight (kg) over time over 12 weeks for each administration group.

### BEST MODE FOR CARRYING OUT THE INVENTION

The present inventors confirmed that cyclo-hispro or a pharmaceutically acceptable salt thereof had an excellent effect of preventing or treating obesity, thereby completing the present invention.

The present invention relates to a method for preventing or treating obesity by using cyclo-hispro or a pharmaceutically acceptable salt thereof.

The present invention relates to a pharmaceutical composition for preventing or treating obesity, comprising cyclo-hispro or a pharmaceutically acceptable salt thereof. In one embodiment, the present invention provides a method for preventing or treating obesity in a subject in need thereof, comprising administering an effective amount of cyclo-hispro or a pharmaceutically acceptable salt thereof to the subject. In another embodiment, the present invention provides a use of cyclo-hispro or a pharmaceutically acceptable salt thereof for the prevention or treatment of obesity.

“Cyclo-hispro (cyclo(his-pro))” is a naturally occurring cyclic dipeptide that has a structure of formula I below and is composed of histidyl and proline. It is also referred to herein as the abbreviation of “CHP.”

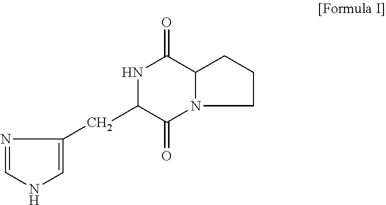 [SMILES: O=C1NC(Cc2c[nH]cn2)C(=O)N2CCCC12]

Cyclo-hispro is found in blood, cerebrospinal fluid (CSF), semen, brain, spinal cord, and gastrointestinal tract of human, and the like. Cyclo-hispro has several biological activities. According to studies, it was found that cyclo-hispro is one of the major metabolites of thyrotropin-releasing hormone (TRH), which is the most produced in the prostate. “Cyclo-hispro” of the present invention may include purified cyclo-hispro.

Specifically, the present invention provides a pharmaceutical composition for preventing or treating obesity, characterized in that the pharmaceutical composition comprises cyclo-hispro or a pharmaceutically acceptable salt thereof as an active ingredient, and the cyclo-hispro or pharmaceutically acceptable salt thereof is administered at an amount of 1 mg to 25 mg per day based on the free base of cyclo-hispro. In one embodiment, the present invention provides a method for preventing or treating obesity in a subject in need thereof, comprising administering to the subject a pharmaceutical composition comprising cyclo-hispro or a pharmaceutically acceptable salt thereof at a dose of 1 mg to 25 mg per day based on the free base of cyclo-hispro.

In addition, the cyclo-hispro or pharmaceutically acceptable salt thereof may be administered at an amount of 3 mg to 20 mg per day based on the free base of cyclo-hispro, and preferably at an amount of 6 mg to 15 mg per day, and most preferably at an amount of 15 mg per day.

The pharmaceutical composition of the present invention may be administered once a day or several times a day, and preferably may be administered once a day.

The pharmaceutical composition of the present invention may be orally or parenterally administered, and preferably may be orally administered.

The pharmaceutical composition of the present invention may be orally administered to an individual via various routes. All methods of administration may be used, and the pharmaceutical composition of the present invention may be administered, for example, by oral administration, intranasal administration, transbronchial administration, intraarterial injection, intravenous injection, subcutaneous injection, intramuscular injection, or intraperitoneal injection. Preferably, the pharmaceutical formulation of the present invention may be administered orally once a day.

The present invention provides a pharmaceutical composition for preventing or treating obesity by reducing body weight and plasma leptin resistance for a long period of time.

The present invention provides a pharmaceutical composition for preventing or treating obesity, comprising cyclo-hispro or a pharmaceutically acceptable salt thereof and zinc as an active ingredient. In one embodiment, cyclo-hispro or a pharmaceutically acceptable salt thereof and zinc may be formulated in one pharmaceutical composition, or may be formulated in separate pharmaceutical compositions. In another embodiment, the present invention provides a method for preventing or treating obesity in a subject in need thereof, comprising administering to the subject cyclo-hispro or a pharmaceutically acceptable salt thereof and zinc. Here, the cyclo-hispro or pharmaceutically acceptable salt thereof may be administered simultaneously, separately, or sequentially with zinc. In addition, the present invention provides a use of cyclo-hispro or a pharmaceutically acceptable salt thereof that is administered in combination with zinc for the prevention or treatment of obesity.

The “zinc” includes, but is not limited to, all of a zinc salt, a zinc ion, a zinc cation, and a zinc anion.

Specifically, the daily dose of cyclo-hispro or a pharmaceutically acceptable salt thereof may be 1 mg to 25 mg based on the free base of cyclo-hispro, and the daily dose of zinc may be 15 mg to 30 mg.

In addition, the daily dose of the zinc may be preferably 23 mg.

The present invention relates to a pharmaceutical composition for preventing or treating obesity, characterized in that the composition comprises 15 mg of cyclo-hispro and 23 mg of zinc and is administered once a day.

The term “pharmaceutically acceptable salt” means a salt that is commonly used in the pharmaceutical field, including hydrochloride, hydrobromide, hydroiodide, hydrofluoride, sulfate, sulfonate, citrate, camphorate, maleate, acetate, lactate, nicotinate, nitrate, succinate, phosphate, malonate, malate, salicylate, phenyl acetate, stearate, formate, fumarate, urea, sodium, potassium, calcium, magnesium, zinc, lithium, cinnamate, methylamino, methanesulfonate, picolinate, p-toluenesulfonate, naphthalenesulfonate, tartrate, triethylamino, dimethylamino, and tri(hydroxymethyl)aminomethane, but not limited thereto.

In order to prepare the pharmaceutical composition of the present invention, appropriate carriers, excipients, and diluents that are commonly used may be further included. In addition, it may be formulated and used in the form of oral preparation such as powders, granules, tablets, capsules, suspensions, emulsions, syrups, and aerosols, external preparations, suppositories, and sterile injectable solutions according to a conventional method.

Carriers, excipients, and diluents that may be included in the composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil, and the like, but are not limited thereto.

In addition, the composition may further comprise diluents or excipients such as conventional fillers, bulking agents, binders, wetting agents, disintegrating agents, surfactants, and the like.

Hereinafter, preferred examples are presented in order to help the understanding of the present invention. However, the following examples are only provided to more easily understand the present invention, and the contents of the present invention are not limited by the following examples.

### EXAMPLE

We conducted a clinical trial designed as randomized, placebo-controlled, double-blind, and two-way crossover on drug interaction in patients with type 2 diabetes mellitus. The clinical trial was conducted for 149 patients over a total of 16 weeks, in which the screening period was 2 weeks, the treatment period was 12 weeks, and the period of the evaluation of safety and follow-up was 2 weeks.

In the screening stage, 64 subjects were selected from 149 participants, and the subjects who met the selection criteria were fasted for 2 hours before and after breakfast each day and subjected to a 2-week evaluation process of measuring and recording blood glucose levels. In the treatment stage, capsules containing placebo or CHP with different doses were randomly administered to 64 selected subjects once a day for 12 consecutive weeks.

In the above experiment, a capsule containing CHP with a different dose or a placebo corresponding thereto was used, and each subject received a two-week dose (18 capsules=14+4) at one visit and was instructed to take 1 capsule before bedtime every day. In this case, the anti-diabetic drugs and other prescribed drugs currently being used were continuously taken. Each subject was assigned at the same rate as follows and administered with a placebo or a compound with a different dose, respectively.
Administration Group A: 3 mg of CHP+23 mg of zinc—16 subjects
Administration Group B: 9 mg of CHP+23 mg of zinc—16 subjects
Administration Group C: 15 mg of CHP+23 mg of zinc—16 subjects
Administration Group D: placebo (control)—16 subjects

### Example 1

Experiment of Change in Concentration of glycated hemoglobin (HbA1c) for Each Administration Group for Different Dose of CHP

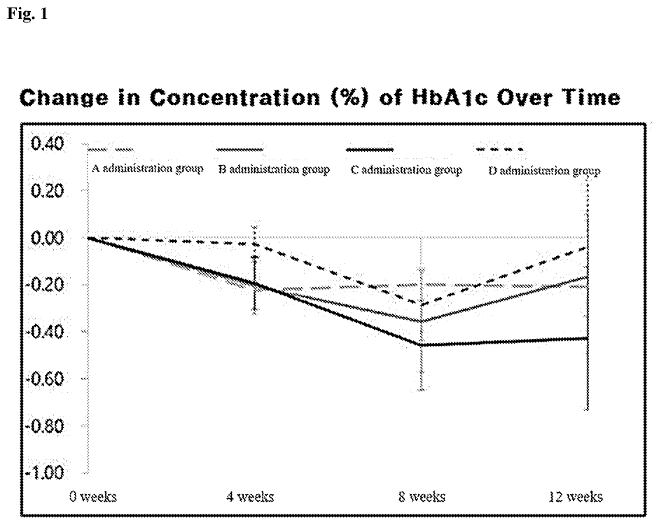

The present inventors observed the change in concentration of HbA1c during the 12-week dosing period. Since the blood glucose level measured at one time point can change due to various factors, the change in glycated hemoglobin (HbA1c) was checked for the purpose of determining the pattern of long-term glycemic control, and the results are shown in Table 1 and FIG. 1.

TABLE 1

| | | Administration | Administration | Administration | Administration |
|---|---|---|---|---|---|
| | | Group A | Group B | Group C | Group D |
| Baseline (Week 0) | Number of | 15 | 16 | 16 | 14 |
| | Subjects | | | | |
| | Average | 8.25 | 8.06 | 8.49 | 8.29 |
| Week 12 | Number of | 12 | 15 | 14 | 13 |
| | Subjects | | | | |
| | Average | 8.04 | 7.90 | 8.08 | 8.21 |
| Change Value (From | Number of | 12 | 15 | 14 | 13 |
| Week 0 to Week 12) | Subjects | | | | |
| | Average | −0.21 | −0.17 | −0.43 | −0.04 |

As shown in Table 1 above, the level of HbA1c at week 12 in Administration Group C was −0.43%, which was the lowest concentration, and the levels of HbA1c at week 12 in Administration Group A and Administration Group B were lower than that of Administration Group D (control).

In addition, as shown in FIG. 1, the level of HbA1c over time was reduced overall in all groups between week 0 and week 4, and in Administration Group C, the level of HbA1c was reduced consistently until week 8, and then there was little change in the level of HbA1c between week 8 and week 12.

As a result, it was confirmed that the level of HbA1c in all patient groups receiving CHP was reduced compared to the control.

### Example 2

Comparative Experiment of Change in Body Weight for Each Administration Group for Different Dose of CHP

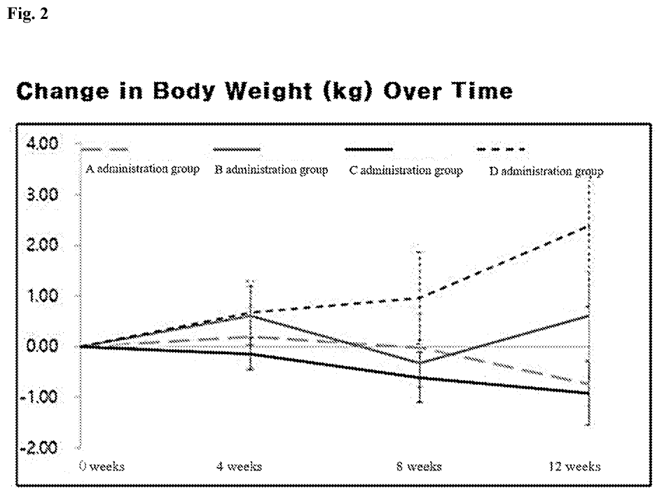

The present inventors observed the change in body weight of subjects through the 12-week experiment, and the results are shown in Table 2 and FIG. 2.

TABLE 2

| | | Administration | Administration | Administration | Administration |
|---|---|---|---|---|---|
| | | Group A | Group B | Group C | Group D |
| Baseline (Week 0) | Number of | 15 | 16 | 16 | 14 |
| | Subjects | | | | |
| | Average | 109.11 | 103.56 | 103.94 | 109.61 |
| Week 12 | Number of | 12 | 15 | 14 | 13 |
| | Subjects | | | | |
| | Average | 108.28 | 105.19 | 104.12 | 113.43 |
| Change Value (From | Number of | 12 | 15 | 14 | 13 |
| Week 0 to Week 12) | Subjects | | | | |
| | Average | −0.73 | 0.61 | −0.92 | 2.38 |

As shown in Table 2, it was confirmed that the body weight at week 12 was increased the most (+2.38 kg) in Administration Group D. On the other hand, it was confirmed that the body weight at week 12 was greatly reduced (−0.92 kg) in Administration Group C. The weight loss of Administration Group A (−0.73 kg) was less than that of Administration Group C, but the body weight was reduced. It was confirmed that the weight gain of Administration Group B (+0.61 kg) was not greater than that of Administration Group D (control).

In addition, as shown in FIG. 2, it was confirmed that the body weight of Administration Group D was increased consistently until week 12, and the body weight of Administration Group C was reduced consistently until week 12.

As a result, it was confirmed that the weight gain in all patient groups receiving CHP was less than the control, or the body weight was reduced.

### Example 3

Comparative Experiment of Improvement of Body Mass Index (BMI) and Leptin Resistance for Each Administration Group for Different Dose of CHP

The present inventors confirmed the improvement of body mass index (BMI) and the improvement of leptin resistance, and the results are shown in Tables 3 and 4.

Leptin is an appetite suppressing protein secreted from adipose cells, and is known as a hormone to act on the brain after being secreted from adipose tissue to suppress appetite and activate metabolism in the body, thereby reducing body weight. In the case of humans, the more obese people (the more adipose tissue), the higher the concentration of leptin in the blood. This indicates the resistance to leptin action.

TABLE 3

| | | Administration | Administration | Administration | Administration |
|---|---|---|---|---|---|
| | | Group A | Group B | Group C | Group D |
| Baseline (Week 0) | Number of | 15 | 16 | 16 | 14 |
| | Subjects | | | | |
| | Average | 37.18 | 36.29 | 37.23 | 37.91 |
| Week 12 | Number of | 12 | 15 | 14 | 13 |
| | Subjects | | | | |
| | Average | 36.45 | 36.59 | 37.48 | 38.72 |
| Change Value (From | Number of | 12 | 15 | 14 | 13 |
| Week 0 to Week 12) | Subjects | | | | |
| | Average | −0.27 | 0.16 | −0.28 | 0.66 |

As shown in Table 3, it was confirmed that the body mass index (BMI) at week 12 was reduced by −0.27 kg/m² and −0.28 kg/m² in Administration Group A and Administration Group C, respectively, but the body mass index (BMI) was increased by 0.66 kg/m² in Administration Group D.

TABLE 4

| | | Administration | Administration | Administration | Administration |
|---|---|---|---|---|---|
| | | Group A | Group B | Group C | Group D |
| Baseline (Week 0) | Number of | 15 | 16 | 16 | 14 |
| | Subjects | | | | |
| | Average | 10.93 | 19.85 | 12.93 | 13.58 |
| Week 12 | Number of | 12 | 15 | 14 | 12 |
| | Subjects | | | | |
| | Average | 9.66 | 9.90 | 10.59 | 11.33 |
| Change Value (From | Number of | 12 | 15 | 14 | 12 |
| Week 0 to Week 12) | Subjects | | | | |
| | Average | 0.59 | −3.42 | −3.39 | −1.87 |

In addition, as shown in Table 4, it was confirmed that the plasma leptin level at week 12 was reduced to a statistically significant level in Administration Group B and Administration Group C compared to the reference value. As a result, it was confirmed that both the body mass index and plasma leptin level were reduced in Administration Group C.

### Example 4

### Result of Comparative Experiment of Evaluation of Safety for Each Dose of Compound 1

The present inventors conducted the evaluation of safety. As a result, mild and moderate side effects that are not related to the CHP were observed, and side effects of increased blood glucose level, dizziness, and ecchymosis were more frequently observed in the control receiving placebo.

As a result, it was confirmed that when the CHP administration groups are compared with the control (placebo), it has an excellent effect as a therapeutic agent for obesity by confirming advantages such as reduced body weight, reduced body mass and reduced leptin level, and reduced side effects.

1. A pharmaceutical composition for preventing or treating obesity, characterized in that the pharmaceutical composition comprises cyclo-hispro or a pharmaceutically acceptable salt thereof, and the cyclo-hispro or pharmaceutically acceptable salt thereof is administered at an amount of 1 mg to 25 mg per day based on the free base of cyclo-hispro.

2. The pharmaceutical composition according to claim 1, characterized in that the cyclo-hispro or pharmaceutically acceptable salt thereof is administered at an amount of 3 mg to 20 mg per day based on the free base of cyclo-hispro.

3. The pharmaceutical composition according to claim 2, characterized in that the cyclo-hispro or pharmaceutically acceptable salt thereof is administered at an amount of 6 mg to 15 mg per day based on the free base of cyclo-hispro.

4. The pharmaceutical composition according to claim 3, characterized in that the cyclo-hispro or pharmaceutically acceptable salt thereof is administered at an amount of 15 mg per day based on the free base of cyclo-hispro.

5. The pharmaceutical composition according to claim 1, characterized in that the composition is administered orally once a day.

6. The pharmaceutical composition according to claim 1, characterized in that the pharmaceutical composition further comprises zinc.

7. The pharmaceutical composition according to claim 6, characterized in that the zinc is administered at an amount of 15 mg to 30 mg per day.

8. The pharmaceutical composition according to claim 7, characterized in that the zinc is administered at an amount of 23 mg per day.

9. The pharmaceutical composition according to claim 1, characterized in that the composition reduces body weight and plasma leptin level.

10. A composition for preventing or treating obesity, characterized in that the composition comprises 15 mg of cyclo-hispro and 23 mg of zinc and is administered once a day.
